(19) United States
(12) Patent Application Publication
(10) Pub. No.: US 2007/0297571 A1
(43) Pub. Date: Dec. 27, 2007

(54) X-Ray Emitter and Method for Generating and Representing X-Ray Images

(75) Inventors: Christian Beckhaus, Darmstadt (DE); Klaus Stockl, Bensheim (DE); Uwe Zeller, Biberach (DE)

Correspondence Address: DYKEMA GOSSETT PLLC, FRANKLIN SQUARE, THIRD FLOOR WEST, 1300 I STREET, NW, WASHINGTON, DC, 20005, US

(73) Assignee: Sirona Dental Systems GmbH, Bensheim (DE)

(21) Appl. No.: 11/587,037

(22) PCT Filed: Apr. 21, 2005
(86) PCT No.: PCT/EP05/51764

§ 371 Date: Oct. 20, 2006

(30) Foreign Application Priority Data
Apr. 23, 2004 (DE) 10 2004 020 370.9

(51) Int. Cl. A61B 6/14; A61B 6/00

(52) U.S. Cl. 378/147

(57) ABSTRACT

An X-ray emitter, particularly a dental X-ray apparatus, includes an X-ray source disposed in a housing, wherein the interior housing has an emission aperture and a diaphragm is provided which delimits the X-rays leaving the emission aperture to form a X-ray fan beam and is disposed at a distance from said emission aperture. In the space between the emission aperture and the diaphragm there are disposed radio-transparent means for deflecting light waves in the visible range, and in or on said housing there is provided an image detection system for the deflected light waves.

### TECHNICAL FIELD

The invention relates to an X-ray emitter and a method for creating and displaying X-ray images.

### DESCRIPTION OF THE RELATED ART

DE 3632878 A1 discloses an apparatus for positioning the head of a patient for the purpose of producing X-ray images, particularly a dental panoramic tomogram, by means of which apparatus the head of the patient is aligned with the display of the recorded silhouette of the patient's head. A video camera and a video monitor are used for this purpose, the stored position being brought into register with the current position on the video monitor. The video camera is adjusted in such a way that its optical axis coincides with that of the central ray of the X-ray emitter. This apparatus appears to be independent of the actual X-ray device.

DE 10148412 A1 discloses a process and an apparatus for imaging the head region of a patient, which apparatus is disclosed for both cephalometric X-ray and photographic images of objects to be X-rayed. The imaging position of the X-ray emitter is adjusted according to the photographic image. Both modes of imaging can be performed concurrently, wherein a photographic camera is arranged in a mirrored arrangement laterally to an X-ray source. The beam path of the photographic camera is deflected by the mirror so as to coincide with the X-ray beam.

It is an object of the invention to provide information, from a single panoramic rotation around an object to be X-rayed, concerning the relationship of the X-ray images and the visible surface of the object.

Another object is to provide a method by means of which not only an X-ray image is produced but also further information concerning the object being examined is acquired and finally displayed.

### SUMMARY AND OBJECTS OF THE INVENTION

This object is achieved with the X-ray emitter according to the invention, which comprises an X-ray source disposed in a housing comprising an emission aperture, a diaphragm being provided, which is located at a distance from the emission aperture and which delimits the X-rays emitted through the emission aperture so as to form an X-ray fan beam. Means for deflecting light waves in the visible range, which are transparent to X-rays, are accommodated in the space between the emission aperture and the diaphragm.

The term “transparent to X-rays” means that the X-rays are not attenuated to such an extent that they become unsuitable for producing the required image of the object to be examined.

Even in the case of tomographic imaging, it is possible, when using the X-ray emitter according to the invention, to produce such images of the visible surface of the object to be X-rayed, as described above. When creating such images, the X-ray emitter must be positioned very close to the object to be X-rayed, as opposed to cephalometric images. Thus the space available for accommodating additional optical components as may be necessary for imaging the visible surface of the object is very small. Accommodating these components, including the required deflection means, in front of the primary diaphragm makes it unnecessary to modify the imaging geometry.

Further advantageous embodiments are evident from the subclaims.

In an advantageous embodiment of the X-ray emitter, the deflection means form a component of the emission aperture. This ensures that the space available is optimally utilized.

In another advantageous embodiment of the X-ray emitter, the deflection means can be pivoted into the beam path of the X-rays. It is then possible to move the deflection means into the beam path only when it is desired to create an image of the visible surface of the object to be X-rayed.

The deflection means advantageously consist of a vacuum-metalized element of plastics material showing low X-ray absorption. In this case, the deflection means can always remain in the beam path and a pivoted mechanism becomes unnecessary.

Furthermore, is has proven to be advantageous if the image recorder is aligned on a plane between the emission aperture and the diaphragm with its imaging direction extending at right angles to, and/or obliquely to, the direction of the X-ray fan beam. The image recorder can thus be accommodated within the housing of the X-ray emitter.

Advantageously, in the X-ray emitter according to the invention, the geometry of the surface image created by light in the range of visible wavelengths is substantially the same as the geometry of the region which is X-rayed. This substantially facilitates the formation of an association between the X-ray image and the visible surface of the object being X-rayed.

It has proven to be particularly advantageous if the area to be X-rayed coincides exactly with the video image delimited by the diaphragm. An automatic computer-aided overlap of the two images is thus made possible.

### BRIEF DESCRIPTION OF THE DRAWINGS

The process according to the invention is explained with reference to the drawings, in which:

FIG. 1 shows a perspective view of an X-ray emitter having an integrated video camera together with the object to be examined,

FIG. 2 shows a cross section of the X-ray emitter illustrated in FIG. 1 taken at the level of emission of the X-ray fan beam,

FIG. 3 shows an emission aperture with integrated deflection means,

FIGS. 4 and 5 show two different embodiments of the adjustable deflection means.

### DETAILED DESCRIPTION OF EXEMPLARY EMBODIMENTS OF THE INVENTION

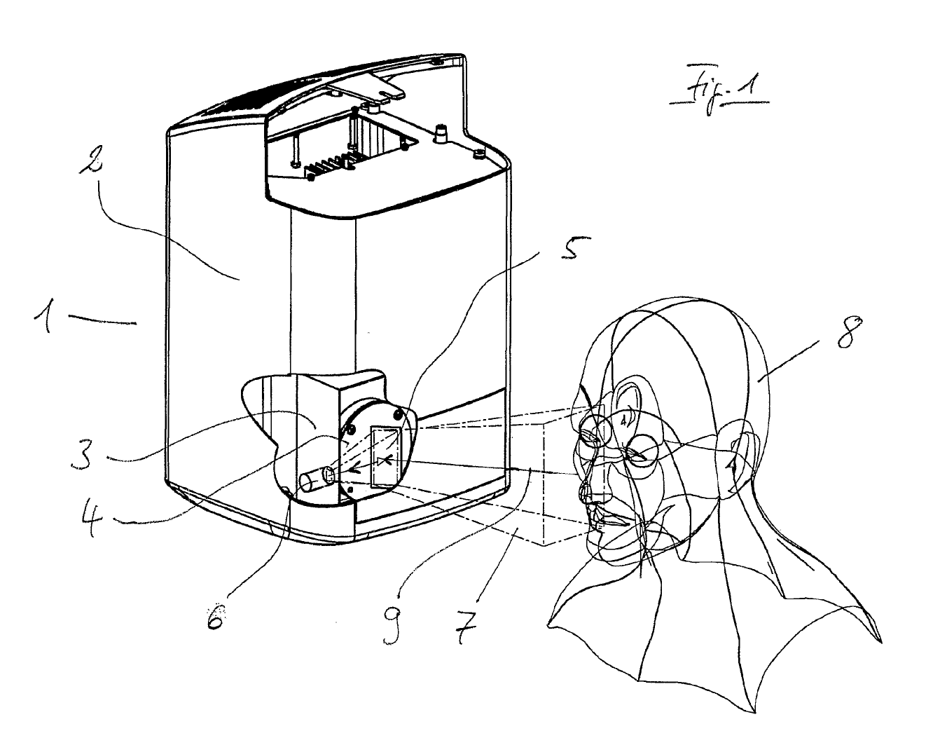

The X-ray emitter 1 shown in FIG. 1 comprises a housing 2, in which the actual X-ray source 3 is disposed. The X-ray source 3 is shielded in such a way that the X-ray is discharged only through an emission aperture. The emission aperture 4 is designed to be transparent to X-rays for this purpose.

Regarded from the X-ray source 3, deflection means 5 in the form of a mirror are located downstream of the emission aperture 4. These deflection means 5 are likewise permeable to X-rays. However, this mirror 5 is substantially opaque to light waves in the optically perceivable range of wavelengths. The mirror 5 is arranged so as to cooperate with an image recording device 6 for light waves in the visible range which is disposed in front of the emission aperture. The X-ray fan beam 7, which is deflected by the mirror 5, is recorded by means of the camera 6. Said X-ray fan beam is directed towards the object to be examined, here a patient 8, and illuminates the surface present thereon. The beam path of the light reflected from the object 8 to the image recording device 6 is illustrated by way of example by means of a central ray 9.

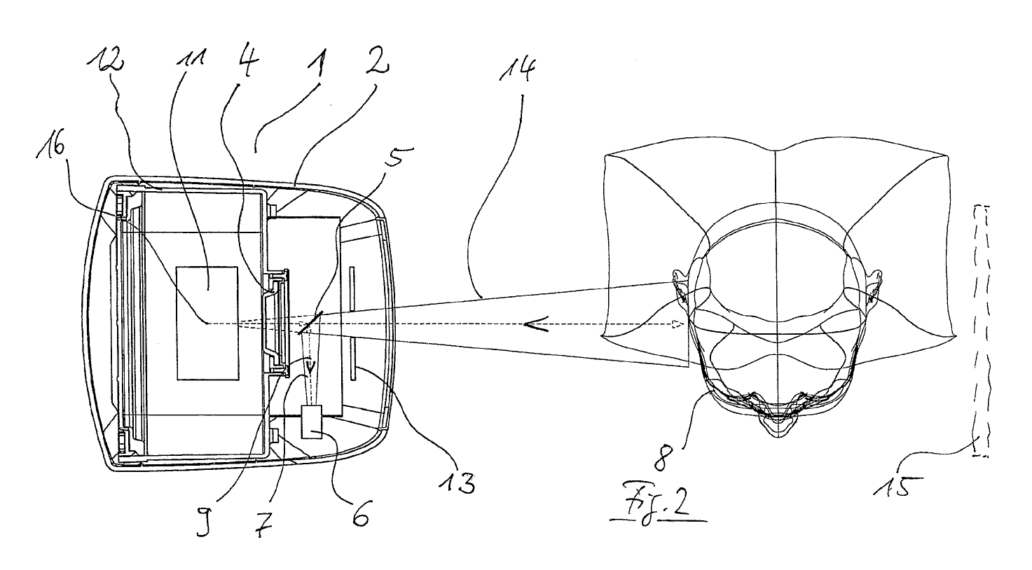

A cross section of the X-ray emitter 1 is shown in FIG. 2. An X-ray tube 11 is located inside the housing, which X-ray tube is shielded from the environment, with the exception of the emission aperture 4, by an internal housing 12, which is impermeable to X-rays. A diaphragm 13 is provided between the emission aperture 4 and the object 8 being examined, which diaphragm delimits the X-ray beam, which is directed towards the object 8 being examined, is produced by the X-ray tube 11, and is discharged through the emission aperture 4 to form a X-ray fan beam.

An X-ray image, which is produced when the X-ray emitter and an sketched image detector 15 for the radiation passing through the object 8 rotates around the object 8 being X-rayed, shows structures, for example, bones, teeth and fillings existing under the surface of the object 8.

The surface of the object to be examined is videographed using the camera 6, which is disposed away from the X-ray fan beam 14 of the X-rays and the beam path of which is aligned by the deflection means 5 such that the beam path 7 with its central ray 9 substantially coincides with the X-ray fan beam 14 and images the same area of the object 8.

At a pre-defined distance of a focus 16 from the diaphragm 13, there is usually sufficient installation space in the emission aperture 4 to accommodate the deflection means 5 and the camera 6. A minimum distance between the focus and the diaphragm is expedient due to the finite expansion of the focus for preventing any disturbing influences of the penumbra on the image detector 15.

In addition, the deflection means can be designed such that they can be moved into and out of the beam path of the X-ray fan beam 14. This can be effected either by means of a motor or manually, for example, using a rod arrangement (not illustrated), which is accessible from outside the housing 2. This is explained in detail below.

FIG. 3 shows an emission aperture 4, of which the outer side remote from the focus is in the form of deflection means for visible light. Such means can take the form of, say, a mirror whose surface is designed as a serrated profile or a fractal lens, affording the possibility of exact focusing.

The emission aperture itself is made of a material permeable to X-rays and the design of the surface located in the beam path affects the quality of the X-rays in the X-ray fan beam by not more than the extent that may be tolerable for the creation of X-ray images.

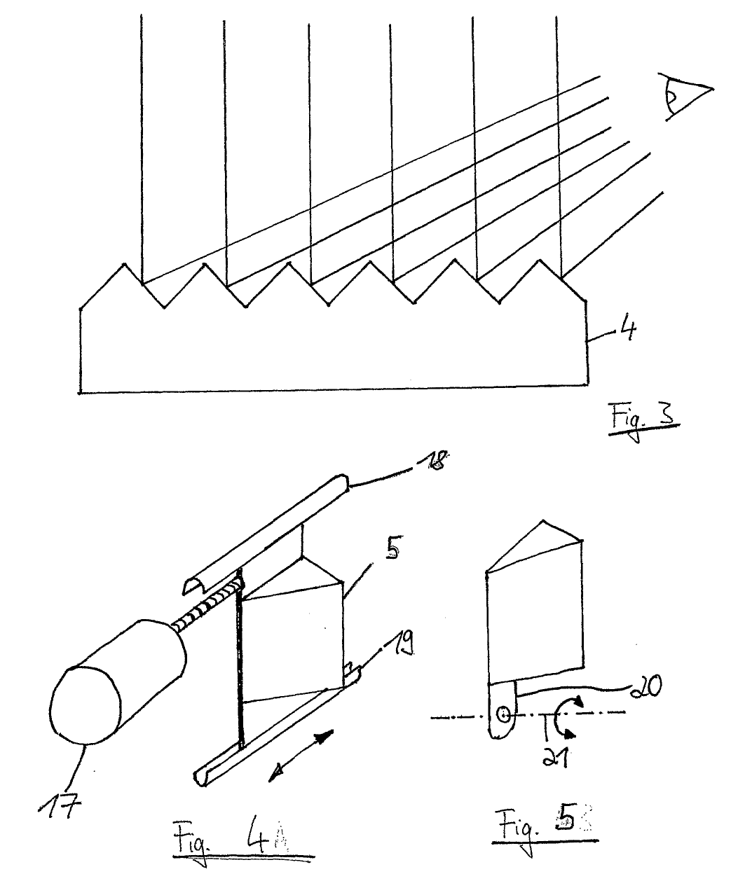

FIGS. 4 and 5 show two different possible embodiments for adjusting insertable deflection means.

The deflection means 5 are inserted along the guides 18, 19 laterally into the beam path by a spindle driven by a motor 17, as illustrated in FIG. 4.

In FIG. 5 a pivoted mechanism is provided for adjusting the deflection means 5, which pivoted mechanism rotates the deflection means 5 fixed to a holder 20 about the rotation axis 21. This rotation can take place either horizontally or vertically depending on the design and the space available.

### LIST OF REFERENCE NUMERALS

1 X-ray emitter
2 Housing
3 X-ray source
4 Emission aperture
5 Deflection means
6 Camera
7 Beam path
8 Patient
9 Central ray
11 X-ray tube
12 Interior housing
13 Diaphragm
14 X-ray fan beam (X-ray beam)
15 Image detector
16 Focus
17 Motor
18 Guide rail
19 Guide rail
20 Holder
21 Rotation axis

1.-7. (canceled)

8. An X-ray emitter, comprising an X-ray source (3) disposed in a housing (2), wherein an interior housing (12) has an emission aperture (4), a diaphragm (13) being provided which delimits the X-rays leaving said emission aperture (4) to form a X-ray fan beam (14) and is disposed at a distance from said emission aperture (4), wherein in the space between said emission aperture (4) and said diaphragm (13) there are disposed means (5) which are transparent to X-rays for deflecting light waves in the visible range, wherein in or on said housing (2) there is provided an image detection system (6) for the deflected light waves, and wherein said deflecting means (5) form a component of said emission aperture (4).

9. An X-ray emitter as defined in claim 8, which comprises part of a dental X-ray apparatus.

10. An X-ray emitter as defined in claim 8, wherein said deflecting means (5) consist of a vacuum-metalized element of plastics material showing low X-ray absorption properties.

11. An X-ray emitter as defined in claim 8, wherein said image detector (6) is oriented on a plane between said emission aperture (4) and said diaphragm (13) in an imaging direction at right angles to, and/or obliquely, to the direction of said X-ray fan beam (14).

12. An X-ray emitter as defined in claim 8, wherein the imaging geometry of the surface registered by means of said image detection (6) for light in the range of visible wavelengths substantially coincides with the geometry of the X-rayed region of the object being X-rayed.

13. An X-ray emitter as defined in claim 8, wherein a region to be X-rayed coincides exactly with the imaging geometry of the surface registered by means of said image detection (6) for light in the range of visible wavelengths.

14. An X-ray emitter, comprising an X-ray source (3) disposed in a housing (2), wherein an interior housing (12) has an emission aperture (4), a diaphragm (13) being provided which delimits the X-rays leaving said emission aperture (4) to form a X-ray fan beam (14) and is disposed at a distance from said emission aperture (4), wherein in the space between said emission aperture (4) and said diaphragm (13) there are disposed means (5) which are transparent to X-rays for deflecting light waves in the visible range, wherein in or on said housing (2) there is provided an image detection system (6) for the deflected light waves, and wherein said deflecting means (5) are adapted to be moved into the optical path of the X-rays.

15. An X-ray emitter as defined in claim 14, which comprises part of a dental X-ray apparatus.

16. An X-ray emitter as defined in claim 14, wherein said deflecting means (5) consist of a vacuum-metalized element of plastics material showing low X-ray absorption properties.

17. An X-ray emitter as defined in claim 14, wherein said image detector (6) is oriented on a plane between said emission aperture (4) and said diaphragm (13) in an imaging direction at right angles to, and/or obliquely, to the direction of said X-ray fan beam (14).

18. An X-ray emitter as defined in claim 14, wherein the imaging geometry of the surface registered by means of said image detection (6) for light in the range of visible wavelengths substantially coincides with the geometry of the X-rayed region of the object being X-rayed.

19. An X-ray emitter as defined in claim 14, wherein a region to be X-rayed coincides exactly with the imaging geometry of the surface registered by means of said image detection (6) for light in the range of visible wavelengths.
